(19) United States
(12) Patent Application Publication
(10) Pub. No.: US 2022/0088103 A1
(43) Pub. Date: Mar. 24, 2022

(54) PEANUT STEM AND LEAF EXTRACT AND PREPARATION METHOD AND USE THEREOF

(71) Applicant: INSTITUTE OF FOOD SCIENCE AND TECHNOLOGY, CHINESE ACADEMY OF AGRICULTURAL SCIENCES, Beijing (CN)

(72) Inventors: Qiang WANG, Beijing (CN); Aimin SHI, Beijing (CN); Lei DENG, Beijing (CN); Rui GUO, Beijing (CN); Hui HU, Beijing (CN); Li LIU, Beijing (CN); Hongzhi LIU, Beijing (CN); Qin GUO, Beijing (CN)

(21) Appl. No.: 16/627,728

(22) PCT Filed: Jul. 18, 2019
(86) PCT No.: PCT/CN2019/096533

§ 371 Date: Dec. 30, 2019

(30) Foreign Application Priority Data
Apr. 19, 2019 (CN) 201910317004.5

(51) Int. Cl. A61K 36/48

(52) U.S. Cl. CPC A61K 36/48; A61K 2236/331

(57) ABSTRACT

A peanut stem and leaf extract contains at least naringenin-4,7′-dimethyl ether, 2′-O-methylisoliquiritigenin, linalool and ferulic acid. The peanut stem and leaf extract can be prepared by pulverizing dried peanut stems and leaves to a micropowder level, followed by water extraction. The water extraction includes placing the pulverized peanut stems and leaves in water at a temperature of 75 to 85° C. for 4 to 5 hours. The preparation method can efficiently extract active substances in peanut stems and leaves, and the peanut stem and leaf extract can effectively improve sleep and treat insomnia.

### CROSS-REFERENCE TO RELATED APPLICATION

The present application claims the priority to the Chinese Patent Application No. 201910317004.5, entitled “Peanut Stem and Leaf Extract and Preparation Method and Use thereof”, filed on Apr. 19, 2019, which is incorporated herein by reference in its entirety.

### TECHNICAL FIELD

The present invention belongs to the field of functional nutritious foods, and particularly relates to a peanut stem and leaf extract and a preparation method and use thereof.

### BACKGROUND ART

According to incomplete statistics, insomnia and sleep problems trouble the world's population. Nearly two-thirds of the population in China still has different levels of sleep problems.

With the accelerated pace of life and the increase in population suffering from insomnia, drug, food and health care products for the treatment of insomnia and sleep improvement have been continuously emerging. However, due to the toxic adverse side effects such as neuro dependence of traditional western medicines, and the disadvantages such as treatment inefficiency and unknown functional ingredients of the traditional Chinese medicines, there has been a lack of sleep improvement products with high public acceptance, clear functional ingredients and high sleep improvement efficiency in China and the global market.

Based on the records of therapeutic effect of peanut stems and leaves in the treatment of insomnia in famous pharmacopoeia such as “South Yunnan Materia Medica”, “Zhejiang Medicinal Plants” and “The Dictionary of Medicinal Plant”, and the practice basis of Chinese medicines for more than 600 years, and the peanut industry status with absolute advantages in China, the present invention discusses a preparation method of a peanut stem and leaf extract with high content of sleep improvement ingredients, and the material composition analysis, functional component screening and confirmation for the peanut stem and leaf extract prepared by the method have been conducted based on the systematic experimental model, high-resolution liquid chromatography-mass spectrometry and advanced electrophysiological technology, and the characteristics of high content and clear effect of sleep improvement active substances have been finally determined.

Therefore, it is of great and far-reaching significance to broaden the products and medicines for improving sleep and treating insomnia, improve the sleep health of Chinese residents, and provide new ideas for the identification and efficacy confirmation of the medicinal and functional active ingredients of traditional Chinese herbal medicines.

### SUMMARY OF THE INVENTION

The purpose of the present invention is to provide a peanut stem and leaf extract comprising at least naringenin-4,7′-dimethyl ether, 2′-O-methylisoliquiritigenin, linalool and ferulic acid.

A peanut stem and leaf extract comprising a high content of active substances having a function of sleep promotion is obtained by extraction and separation in the present invention; wherein the peanut stem and leaf extract contains naringenin-4,7′-dimethyl ether, 2′-O-methylisoliquiritigenin, ferulic acid and linalool that having a potential functional activity for promoting sleep identified according to high resolution liquid chromatography-mass spectrometry;

wherein the content of the naringenin-4,7′-dimethyl ether is not less than 110 mg/kg;

the content of the 2′-O-methylisoliquiritigenin is not less than 32 mg/kg;

the content of the linalool is not less than 44 mg/kg; and

the content of ferulic acid is not less than 175 mg/kg.

Preferably, the content of naringenin-4,7′-dimethyl ether is 114.75±3.42 mg/kg; the content of 2′-O-methylisoliquiritigenin is 32.40±0.02 mg/kg; the content of linalool is 44.44±0.37 mg/kg; and the content of ferulic acid is 189.86±11.40 μg/kg.

Another purpose of the present invention is to provide a method for preparing the above-mentioned peanut stem and leaf extract, the method comprises the following step:

dried peanut stems and leaves are pulverized to a micropowder level, and then extracted by water extraction.

In order to increase the extraction ratio and the concentration of the extracted active substances, the present invention further improves the conditions and parameters of the water extraction on the basis of optimizing the water extraction.

Wherein, the present invention preferably adopts the stems and leaves of the harvest period, especially the stems and leaves in a cyan and unwithered state; and on the basis of selecting the stems and leaves of this state, the manner of extraction is optimized.

Wherein, the water extraction method comprises the following step: placing the pulverized peanut stems and leaves in water at a temperature of 75 to 85° C. for 4 to 5 hours.

Wherein, the micropowder level is achieved by the following step: the dried peanut stems and leaves are subjected to ultrafine-pulverization such that the particle size of the material particles is 1 to 100 μm.

Preferably, the drying is performed as follows: drying the fresh peanut stems and leaves to a moisture content of 10% to 15%.

More preferably, the drying is carried out in a manner of natural drying. For example, the fresh peanut stems and leaves of the harvest period are selected, washed and dried under ventilation and sunshine.

Preferably, the solid-liquid ratio of the peanut stems and leaves to water (weight ratio) is 10-20:1.

Preferably, in the water extraction method, the extraction is cycled for 1 to 4 times; preferably 2 times. That is, the extraction is carried out at this temperature for 4 to 5 hours and cycled for 2 times.

Wherein, after the end of the extraction, the method further comprises drying which may be low temperature drying under reduced pressure, and further preferably the drying is carried out as follows: placing the double-effect concentrated extract liquid on a desiccant material plate and drying at a temperature of 40 to 50° C. and a vacuum degree of −0.1 to −0.5 Mpa for 24 to 48 h.

The present invention provides a preferred solution, and the preparation method comprises the following steps:

1) the fresh peanut stems and leaves are dried to a moisture content of 10% to 15%, and then the dried peanut stems and leaves are pulverized to a micropowder level;

2) the pulverized peanut stems and leaves are added to water at a temperature of 75 to 85° C. at a solid-liquid ratio of 10-20:1, and extracted for 4 to 5 hours, and the step 2) is cycled for 2 times;

3) the extract liquid of step 2) is dried by a negative-pressure and low-temperature dryer, and the temperature of the drying oven is controlled at 40 to 50° C., and the vacuum degree is controlled at −0.1 to −0.5 Mpa.

The present invention also provides a pharmaceutical composition or a health care composition, wherein the peanut stem and leaf extract is used as an active substance, and a conventional auxiliary material is added to form a dosage form acceptable to the mass consumer groups.

Preferably, the dosage form may be one of a tablet, a capsule, a powder, a mixture, a pill, a granule, a syrup, an emplastrum, a suppository, an aerosol, an ointment or an injection.

The auxiliary material added is one or more of a filler, a disintegrating agent, a lubricant, a suspending agent, a binder, a sweetener, a corrigent, a preservative, and a matrix.

Wherein, the filler comprises: starch, pregelatinized starch, lactose, mannitol, chitin, microcrystalline cellulose, sucrose and the like.

Wherein, the disintegrating agent comprises: starch, pregelatinized starch, microcrystalline cellulose, sodium carboxymethyl starch, crosslinked polyvinylpyrrolidone, low substituted hydroxypropyl cellulose, croscarmellose sodium and the like.

Wherein, the lubricant comprises: magnesium stearate, sodium lauryl sulfate, talc powder, silica and the like.

Wherein, the suspending agent comprises: polyvinylpyrrolidone, microcrystalline cellulose, sucrose, agar, hydroxypropyl methylcellulose and the like.

Wherein the binder comprises: starch slurry, polyvinylpyrrolidone, hydroxypropyl methylcellulose and the like.

Wherein, the sweetener comprises: sodium saccharin, aspartame, sucrose, cyclamate, glycyrrhetinic acid and the like;

Wherein the corrigent comprises: a sweetener and various essences;

Wherein, the preservative comprises: paraben, benzoic acid, sodium benzoate, sorbic acid and salts thereof, benzalkonium bromide, chlorhexidine acetate, eucalyptus oil and the like; the matrix comprises: PEG6000, PEG4000, insect wax and the like.

The peanut stem and leaf extract and the pharmaceutical composition or the health care composition provided by the present invention have a remarkable effect in promoting sleep. They can effectively improve sleep and treat insomnia.

The BAL b/c mice is used as a model in the present invention, and the electrophysiological technology is adopted, wherein electrodes are implanted in the brain of the mice, and the brain wave changes of mice after drug administration is real time 24-hour monitored for 14 days, thereby determining that the functional active substances in the peanut stem and leaf extract provided by the present invention have a significant sleep promoting function.

### BRIEF DESCRIPTION OF THE DRAWINGS

FIG. 1 is quantitative LC-MS spectrums of four biologically active substances in Experimental Example 1;

FIG. 2 is a graph showing the effect of the peanut stem and leaf extract on the 24-hour sleep time of mice in Experimental Example 2;

FIG. 3 is a graph showing the effects of four functional active substances on 24-hour sleep of mice in Experimental Example 2.

### SPECIFIC MODES FOR CARRYING OUT THE EMBODIMENTS

The following Examples are intended to illustrate the present invention, but are not intended to limit the scope of the present invention.

### Example 1

The present Example provides a method for preparing a peanut stem and leaf extract, and the method comprises the following steps:

1) the fresh peanut stems and leaves of the harvest period were selected, washed and dried under ventilation and sunshine until a moisture content of 10% to 15%, then the dried peanut stems and leaves were subjected to ultrafine-pulverization such that the particle size of the material particles was 1 to 100 μm;

2) 100 g of the pulverized peanut stem and leaf powder were taken and placed into an extracting tank, 10 times by weight of water were added for extraction at a temperature of 80 to 85° C. for 4 to 5 hours, and the extraction was cycled for 2 times;

3) the extract liquid was dried by a negative-pressure and low-temperature dryer, and the temperature of the drying oven was controlled at 40 to 50° C., and the vacuum degree was controlled at −0.1 to −0.5 Mpa.

The extraction ratio of the peanut stem and leaf extract prepared in the present Example was calculated to be 21.8%.

### Example 2

The present Example provides a method for preparing a peanut stem and leaf extract, and the method comprises the following steps:

1) the fresh peanut stems and leaves of the harvest period were selected, washed and dried under ventilation and sunshine until a moisture content of 10% to 15%, then the dried peanut stems and leaves were subjected to ultrafine-pulverization such that the particle size of the material particles was 1 to 100 μm;

2) 100 g of the pulverized peanut stem and leaf powder were taken and placed into an extracting tank, 15 times by weight of water were added for extraction at a temperature of 80 to 85° C. for 4 to 5 hours, and the extraction was cycled for 2 times;

3) the extract liquid was dried by a negative-pressure and low-temperature dryer, and the temperature of the drying oven was controlled at 40 to 50° C., and the vacuum degree was controlled at −0.1 to −0.5 Mpa.

The extraction ratio of the peanut stem and leaf extract prepared in the present Example was calculated to be 25.8%.

### Example 3

The present Example provides a method for preparing a peanut stem and leaf extract, and the method comprises the following steps:

1) the fresh peanut stems and leaves of the harvest period were selected, washed and dried under ventilation and sunshine until a moisture content of 10% to 15%, then the dried peanut stems and leaves were subjected to ultrafine-pulverization such that the particle size of the material particles was 1 to 100 μm;

2) 100 g of the pulverized peanut stem and leaf powder were taken and placed into an extracting tank, 20 times by weight of water were added for extraction at a temperature of 80 to 85° C. for 4 to 5 hours, and the extraction was cycled for 2 times;

3) the extract liquid was dried by a negative-pressure and low-temperature dryer, and the temperature of the drying oven was controlled at 40 to 50° C., and the vacuum degree was controlled at −0.1 to −0.5 Mpa.

The extraction ratio of the peanut stem and leaf extract prepared in the present Example was calculated to be 22.1%.

### Example 4

The present Example provides a method for preparing a peanut stem and leaf extract, which is the same as that of Example 1 except that “peanut stems and leaves of the harvest period” was replaced by “green and tender peanut stems and leaves of the late growing period”.

The extraction ratio of the peanut stem and leaf extract prepared in the present Example was 15%.

### Example 5

The present Example provides a method for preparing a peanut stem and leaf extract, which is the same as that of Example 1 except that the extract liquid was dried by a spray drying apparatus. The extraction ratio of the peanut stem and leaf extract of the present Example was 20% to 25%.

However, detection showed that the contents of functional active substances contained in the peanut stem and leaf extract were significantly reduced, and the specific contents were as follows: naringenin-4,7′-dimethyl ether at a content of 85 mg/kg; 2′-O-methylisoliquiritigenin at a content of 24 mg/kg; linalool at a content of no less than 12 mg/kg; and/or ferulic acid at a content of 154 mg/kg.

### Example 6

The present Example provides a method for preparing a peanut stem and leaf extract, which is the same as that of Example 1 except that vacuum freeze-drying was used for drying. The extraction ratio of the peanut stem and leaf extract and the content of the four biologically active substances in the present Example did not change significantly, but the drying time increased to 48 to 72 hours.

### Experimental Example 1

1. The biologically active substances in the peanut stem and leaf extract obtained in Example 1 were identified as follows:

1) extraction of active substances: 10 g of the peanut leaf and leaf water-extract powder were weighed and subjected to Soxhlet extraction with ethyl acetate, petroleum ether (40° C. to 60° C.), and n-butyl alcohol, respectively. After extraction, the solvent was collected and subjected to rotary evaporation under reduced pressure by using a vacuum rotary evaporator until the residue is almost dry. 25 ml of chromatographic grade acetonitrile was taken for redissolution, the resultant was wrapped by tin foil paper, and stored at an ultra-low temperature of −80° C. for use;

2) identification of active substances: a UPLC-QTOF-MS detection equipment and a UNIFI data analysis platform were used to analyze the composition of the extracts obtained by different extraction methods. The conditions were as follows: column temperature was 45° C., sample temperature was 5° C.; water (containing 0.1% formic acid) was used as A phase of the mobile phase, acetonitrile (containing 0.1% formic acid) was used as B phase of the mobile phase; flow rate during injection was 0.6 ml/min, injection volume was 10 μL; weak eluent: 90/10 (water/acetonitrile), and 600 μL per column loading;

Elution gradient was shown in the table below.

TABLE 1

| Time (min) | % A | % B | Curvature |
|---|---|---|---|
| 0 | 90 | 10 | 6 |
| 5 | 80 | 20 | 6 |
| 10 | 65 | 35 | 6 |
| 21 | 55 | 45 | 6 |
| 23 | 45 | 55 | 6 |
| 27.5 | 25 | 75 | 6 |
| 30 | 10 | 90 | 6 |

Positive ion and sensitivity analysis mode was selected for QTOF-MS data acquisition; ion mass-to-charge ratio was m/z 50-1000; corona voltage was 2.1 kV, cone voltage was 40 V, a temperature of ion source was 120° C., and collision energy was 15 to 45V. MSE full scan data acquisition mode with a scan interval of 0.2 seconds was selected.

Information on the biologically active substances in the peanut stem and leaf extract prepared in Example 1: it includes 139 biologically active substances.

2. The contents of functional active substances were detected quantitatively by the following method.

The prepared peanut stem and leaf water-extract powder was dissolved in pure water, and the concentration was determined according to the recommended injection concentration for high resolution mass spectrometry, and was not higher than 1 mg/kg in principle. The contents of naringenin-4,7′-dimethyl ether, 2′-O-methylisoliquiritigenin, ferulic acid, and linalool in peanut stems and leaves were analyzed quantitatively by using gas chromatography/ultra-high performance liquid chromatography coupled with triple quadrupole tandem mass spectrometry with high resolution and accuracy. In order to fully guarantee the accuracy and credibility of the test results, the content of linalool was determined by using gas chromatography-mass spectrometry (GC-QQQ-MS), and the contents of naringenin-4,7′-dimethyl ether, 2′-O-methylisoliquiritigenin and ferulic acid were determined by using ultra high performance liquid chromatography-mass spectrometry (UHPLC-QQQ-MS) in the test. The selection of the method was mainly based on the volatility of the compounds and the identification principle of the gas/liquid chromatography-mass spectrometer. Because of the high volatility of linalool, the test for linalool was carried out by using GC-MS. The relevant test parameters as well as the contents of the four functional active substances were shown in the table below.

TABLE 2

| | Quanti- | | | |
|---|---|---|---|---|
| | tative | Standard | Correlation | |
| Components | ion | curve | coefficient | Content |
| naringenin-4,7′- | 302.2→ | Y = 2.92 * | 0.9998 | 114.75 ± 3.42 |
| dimethyl ether | 284.0 | X + 1563.41 | | mg/kg |
| 2′-O-methyl- | 269.1→ | Y = 1.28 * | 0.9999 | 32.40 ± 0.02 |
| isoliquiritigenin | 135.6 | X + 3.29 | | mg/kg |
| ferulic acid | 192.9→ | Y = 3.64 * | 0.9997 | 44.44 ± 0.37 |
| | 133.8 | X − 8.57 | | mg/kg |
| linalool | 93.0 | Y = 94.87 * | 0.9998 | 189.86 ± 11.40 |
| | | X + 1469.07 | | μg/kg |

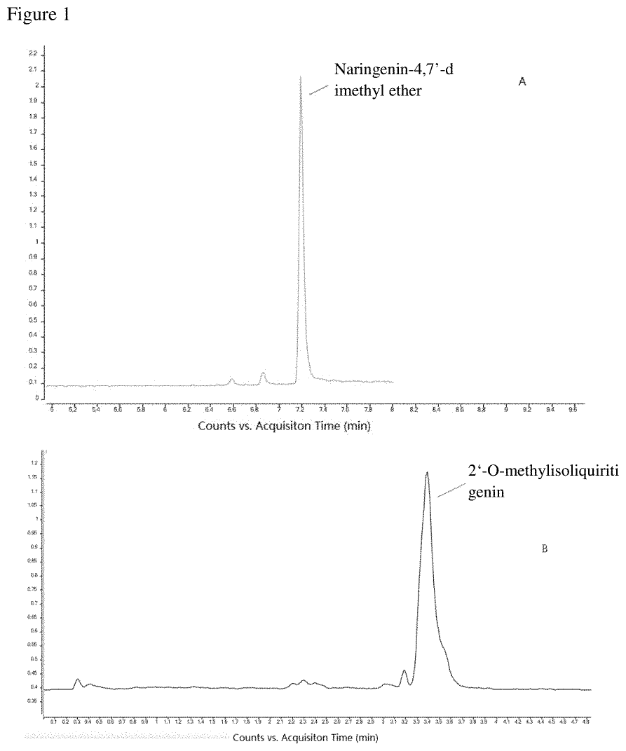

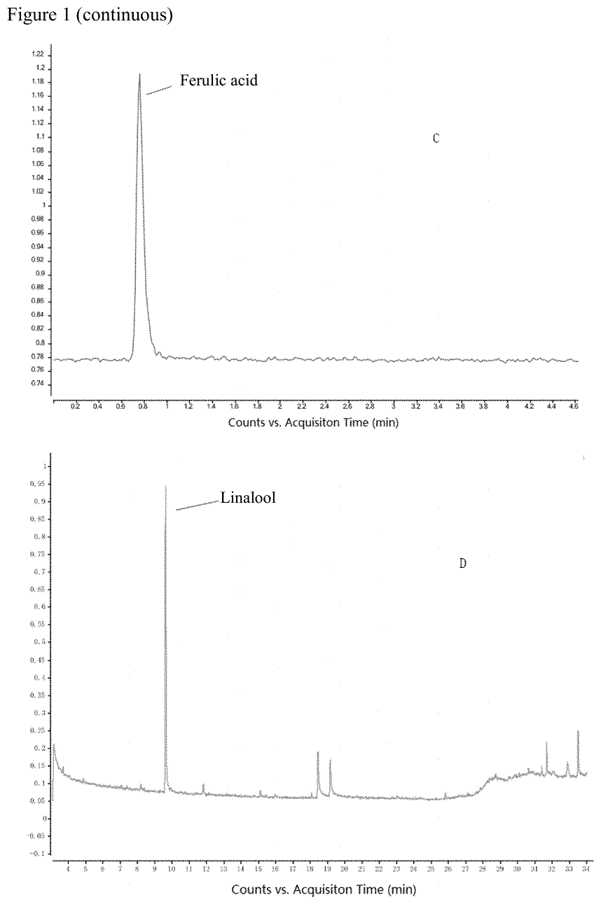

Quantitative LC-MS spectrums of four biologically active substances (A: naringenin-4,7′-dimethyl ether, B: 2′-O-methylisoliquiritigenin, C: ferulic acid, D: linalool) were shown in FIG. 1.

### Experimental Example 2

Experiment of the effect of the peanut stem and leaf extract prepared in Example 1 on sleep of mice

I. Experimental Materials

1 mL syringe (Jiangsu Yuyang Medical Devices Co., Ltd.); Sleepsign sleep analysis software (Kissei Comtec Co., Ltd., Japan); stainless steel small screw M1.0*2 (Halleluya Precision Metal Products Co., Ltd.); MP150 multi-channel physiological signal record analysis system (Biopac company, USA); mouse adapter (Shenzhen RuiWoDe Life Technology Co., Ltd.); high speed cranial drill (Shenzhen RuiWoDe Life science and technology Co., Ltd.); dental cement (Shanghai Second Medical Zhangjiang Biomaterial Co., Ltd.); denture base resins (Shanghai Second Medical Zhangjiang Biomaterial Co., Ltd.); recording cable and electrode (Dongguan Yaobo Electronics Co., Ltd.); tissue glue (Japan Toagosei company); other related reagents (TaKaRa company, Japan); and silver wire for electrode (Cooner Wire company, USA).

II. Experimental Method

1. Test Drug

The peanut stem and leaf extract prepared according to the method of Example 1 was used as an experimental drug, and the standard drugs were administered to the mice according to the identified contents of the substances such as naringenin-4,7′-dimethyl ether.

2. Experimental Animals and Grouping

The experiment was divided into a control group, extract groups (three groups of low, medium and high doses), a positive control group and a functional factor group (four functional active substances). Male Bal b/c mice were used, 6 mice for each group.

3. Timeline of the Experiment and Specific Steps:

The mice were anesthetized at abdominal cavity with chloral hydrate (4%) at 0.10 mL/10 g. After the pain reflex disappeared, the mice were fixed on a mouse brain stereotactic adapter, and the hair on the head of the mice was cut off and 75% alcohol was used to disinfect the skin, the skin was cut along the midline of the skull, and the subcutaneous tissue was separated to fully expose the skull. A hole was drilled through the skull by a cranial drill at the position of 1.0 mm at the front of the anterior skull and 1.5 mm at the left side of the midline of the skull, and the skull was drilled by a cranial drill at the position of 1.0 mm at the front of the posterior fontanelle and 1.5 mm at the left side of the midline of the skull (note that: do not break the meninges, a suitable diameter of the drill was 0.8 mm, and two holes can also be on the right side of the midline of the skull, but both two holes need to be on the same side). The electroencephalo-graph (EEG) electrodes were fixed on the skull with tissue glue and dental cement successively, and the EEG electrode was connected to a screw (fixed in the hole drilled on the skull) to touch the cerebral dura mater by a silver wire to record cortical EEG activity. The two silver wires connected to the myoelectric electrodes were inserted into the muscles on both sides of the neck of the mouse to record the myoelectric activity. The electrode base was then fixed to the skull with tissue glue and dental cement. Mice were injected with penicillin after surgery (measuring 80,000 units/day for three consecutive days). The mice were raised separately in a shielding box with constant temperature, constant humidity, sound insulation and electrostatic shielding function during the recovery period after surgery. The cables for recording EEG and myoelectricity were connected to the electrodes on the head of the mouse on the second day after surgery, and the mice were allowed to adapt for 7 days. The light illumination in the shielding box was from 8:00 am to 8:00 pm. The EEG and electromyograph of mice that recovered and adapted to the experimental environment began to be recorded from 8:00 in the morning for 24 hours, and this day was recorded as D0. This data was used as a self-control, and then the drug was intragastrically administered from D1 to D14 before 8:00 am. The EEG and electromyograph were recorded on the D7 and D14, and the brain tissue was taken after gavage at 8:00 in the morning on D15.

4. Experimental Results

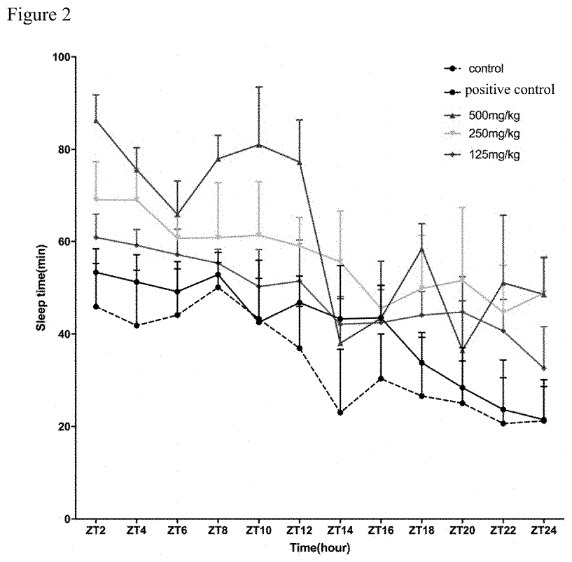

The effect of the peanut stem and leaf extract on the 24-hour sleep time of mice was shown in FIG. 2; wherein, the total sleep time of the mice can be significantly increased by 125 mg/kg (BW), 250 mg/kg (BW), and 500 mg/kg (BW) of extracts, and the effect increases as the administration time and the concentration of the administration increases.

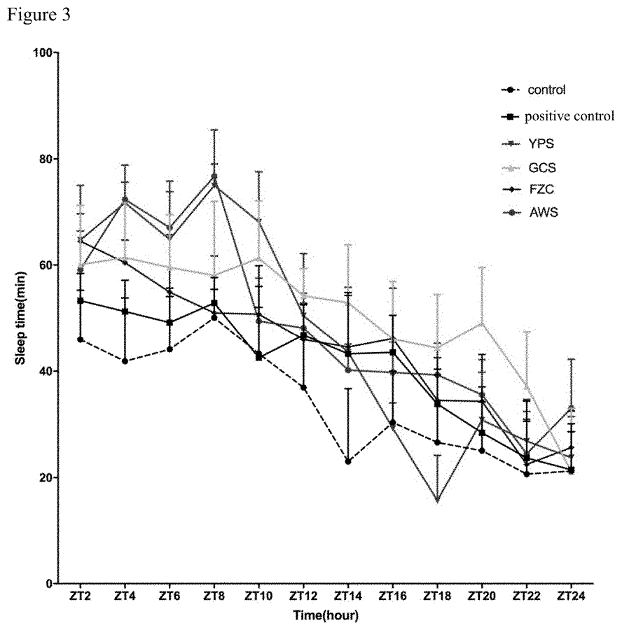

The effects of four functional active substances (YPS: naringenin-4′,7-dimethyl ether, GCS: 2′-O-methylisoliquiritigenin, AWS: Ferulic acid, FZC: linalool) on 24-hour sleep of mice were shown in FIG. 3; wherein, naringenin-4,7′-dimethyl ether, 2′-O-methylisoliquiritigenin, ferulic acid and linalool can significantly improve the sleep time of mice.

Although the present invention is described in detail using general description, specific embodiments and experiments hereinbefore, it is obvious to a person skilled in the art that some modifications or improvements can be made based on the present invention. Hence all these modifications or improvements made on the basis of not deviating from the spirit of the present invention fall into the protection scope claimed in the present invention.

### INDUSTRIAL APPLICABILITY

The present invention provides a peanut stem and leaf extract. The peanut stem and leaf extract comprises at least naringenin-4,7′-dimethyl ether, 2′-O-methylisoliquiritigenin, linalool and ferulic acid. The invention also provides a preparation method of the peanut stem and leaf extract, wherein the peanut stem and leaf extract is prepared by the following method: the dried peanut stems and leaves are pulverized to a micropowder level, and then extracted by water extraction; wherein the water extraction comprises the following step: placing the pulverized peanut stems and leaves in water at a temperature of 75 to 85° C. for 4 to 5 hours. The preparation method provided by the present invention can efficiently extract active substances in peanut stems and leaves, and the peanut stem and leaf extract can effectively improve sleep and treat insomnia, and has high economic value and good application prospect.

1. A peanut stem and leaf extract, wherein the extract comprises at least naringenin-4,7′-dimethyl ether, 2′-O-methylisoliquiritigenin, linalool and ferulic acid.

2. The peanut stem and leaf extract according to claim 1, wherein the content of the naringenin-4,7′-dimethyl ether is not less than 110 mg/kg; and/or the content of 2′-O-methylisoliquiritigenin is not less than 32 mg/kg; and/or the content of the linalool is not less than 44 mg/kg; and/or the content of ferulic acid is not less than 175 mg/kg.

3. A method for preparing a peanut stem and leaf extract, wherein dried peanut stems and leaves are pulverized to a micropowder level, and then extracted by water extraction,
wherein the water extraction method comprises: placing the pulverized peanut stems and leaves in water at a temperature of 75 to 85° C. for 4 to 5 hours.

4. The preparation method according to claim 3, wherein the peanut stems and leaves are stems and leaves of the harvest period, and the stems and leaves are in a cyan and unwithered state.

5. The preparation method according to claim 4, wherein the drying is carried out as follows: drying the fresh peanut stems and leaves to a moisture content of 10% to 15%;
more preferably, the drying is carried out in a manner of natural drying.

6. The preparation method according to claim 4, wherein the solid-liquid ratio of the peanut stems and leaves to water is 10-20:1.

7. The preparation method according to claim 4, wherein in the water extraction, the extraction is cycled for 1 to 4 times; preferably 2 times.

8. The preparation method according to claim 4, wherein, after the end of the extraction, the method further comprises drying which is carried out as follows: placing the extract liquid on a desiccant material plate and drying at a temperature of 40 to 50° C. and a vacuum degree of −0.1 to −0.5 Mpa.

9. A medicine or health care product for promoting sleep, comprising the peanut stem and leaf extract according to claim 1.

10. The medicine or health care product according to claim 9, wherein the medicine or health care product is in a form of any one of a tablet, a capsule, a powder, a mixture, a pill, a granule, a syrup, an emplastrum, a suppository, an aerosol, an ointment, or an injection.

11. The preparation method according to claim 5, wherein the solid-liquid ratio of the peanut stems and leaves to water is 10-20:1.

12. The preparation method according to claim 5, wherein in the water extraction, the extraction is cycled for 1 to 4 times; preferably 2 times.

13. The preparation method according to claim 6, wherein in the water extraction, the extraction is cycled for 1 to 4 times; preferably 2 times.

14. The preparation method according to claim 5, wherein, after the end of the extraction, the method further comprises drying which is carried out as follows: placing the extract liquid on a desiccant material plate and drying at a temperature of 40 to 50° C. and a vacuum degree of −0.1 to −0.5 Mpa.

15. The preparation method according to claim 6, wherein, after the end of the extraction, the method further comprises drying which is carried out as follows: placing the extract liquid on a desiccant material plate and drying at a temperature of 40 to 50° C. and a vacuum degree of −0.1 to −0.5 Mpa.

16. The preparation method according to claim 7, wherein, after the end of the extraction, the method further comprises drying which is carried out as follows: placing the extract liquid on a desiccant material plate and drying at a temperature of 40 to 50° C. and a vacuum degree of −0.1 to −0.5 Mpa.
